(19) United States
(12) Patent Application Publication
(10) Pub. No.: US 2003/0092168 A1
(43) Pub. Date: May 15, 2003

(54) Method for producing an extract rich in nucleic acids from a plant material

(76) Inventors: Christian Lubrano, Issy Les Moulineaux (FR); Georges-Olivier Maillet, Malakoff (FR); Frederique Poirier, Issy Les Moulineaux (FR)

Correspondence Address: YOUNG & THOMPSON, 745 SOUTH 23RD STREET 2ND FLOOR, ARLINGTON, VA, 22202

(21) Appl. No.: 10/277,052

(22) Filed: Oct. 22, 2002

(30) Foreign Application Priority Data
Oct. 22, 2001 (FR) 01 13619

(51) Int. Cl. C12N 1/08

(52) U.S. Cl. 435/270

(57) ABSTRACT

The invention relates to a method for producing an aqueous extract rich in nucleic acids from a plant material, in particular from plant embryos or from seeds rich in DNA or in RNA, which consists in:

extracting, in an aqueous medium, the plant material in the presence of cellulolytic enzymes at an initial pH of 9 to 13,

separating the plant material in order to recover an aqueous extract,

treating the extract with a protease,

separating the insolubles in order to recover a purified aqueous extract.

The present invention relates to a method for producing an extract rich in nucleic acids (DNA and/or RNA) from a plant material and in particular from plant embryos or from seeds rich in nucleic acids, mainly for application in cosmetics.

WO 84/03835 describes a cosmetic composition intended for avoiding skin aging and for bringing about the regeneration of skin cells. This composition contains an aqueous extract of plant embryos enriched with DNA. This extract is obtained from wheat and/or soybean germs using a large number of treatments, including treatments with an anionic detergent and various solvents including chloroform and octanol.

The present invention aims to provide a method for producing an extract rich in nucleic acids from a plant material such as wheat germs, which is simple to carry out and therefore industrializable and which involves nondenaturing treatments as may be the case with detergents and various organic solvents and at the same time not leaving traces of harmful products, and therefore leading to a safer final product.

The subject of the present invention is thus a method for producing an aqueous extract rich in nucleic acids from a plant material, in particular from plant embryos or from seeds rich in DNA or in RNA, which consists in:

extracting, in an aqueous medium, the plant material in the presence of cellulolytic enzymes at an initial pH of 9 to 13,

separating the plant material in order to recover an aqueous extract,

treating the extract with a protease,

separating the insolubles in order to recover a purified aqueous extract.

The plant material used preferably consists of plant embryos such as wheat or soybean germs or alternatively seeds rich in DNA or RNA and is preferably used in ground form.

A size, after grinding, of less than 300 microns is preferred.

For the treatment with cellulolytic enzymes, it is possible to use in particular pectinases or cellulases.

The plant material, in particular wheat germs, is advantageously used in a plant material/water ratio of from 5 to 25% weight/volume.

Effective extraction of the nucleic acids is obtained by setting the initial pH at a relatively basic value (9 to 13). During the extraction, the pH decreases toward neutrality, which constitutes an optimum for the action of cellulolytic enzymes.

The treatment temperature is advantageously less than 70° C. and is preferably 20 to 60° C. The duration of treatment is advantageously from 1 to 2 hours and this treatment is advantageously carried out, after rapid dispersion of the mixture, with slow stirring.

The separation of the crude aqueous extract from the plant material is carried out using conventional devices, such as filters, centrifugal machines, vibrating screens, and preferably using a centrifugal settler.

The crude extract thus separated is then treated with a protease so as to both promote subsequent separation of insoluble products and hydrolyze proteins which are not very soluble. This treatment makes it possible, in particular, to then remove most of the starch still present in the crude extract and thereby to obtain, if desired, a final dry extract which can be more easily resolubilized, a very,precious advantage for subsequent incorporation into a cosmetic composition.

The quantity of protease used depends on the enzymatic activity specific to the commercial enzyme used.

In general, the protease treatment is carried out at a pH of 5 to 8 under temperature, duration and stirring conditions similar to those used for the initial extraction.

The separation of the insolubles may be carried out according to conventional methods. It is however preferable to use ultrafiltration or tangential microfiltration using membranes having a 50 Kd cut-off at 0.4 μm or even better at 0.2 μm.

The purified aqueous extract may be used directly. In general, it is however freeze-dried or spray-dried for subsequent use.

The freeze-dried product thus obtained may contain:

| DNA | 0.1 to 1% | by weight |
|---|---|---|
| RNA | 0.2 to 1.5% | by weight |
| Carbohydrates | 50 to 70% | by weight |
| Proteins | 20 to 40% | |
| Minerals | 5 to 10% | |
| Vitamin B | 5 to 50 | mg/100 g |
| Lipids | <1% | |

It has been shown that the purified extract,

protects cultured skin cells when they are irradiated with UV radiation,

restores the replication potential of the cells after UV irradiation.

Such an extract is therefore of great interest in cosmetic compositions.

The purified extract may be used in cosmetic compositions in amounts of 0.01 to 1%, corresponding to DNA amounts of the order of 0.1 to 10 ppm and RNA amounts of the order of 0.5 to 50.ppm.

An example of production of an extract obtained from wheat germs will be given below.

### EXAMPLE

Micronized powdered wheat germ having a size of 50 to 300 μm is used as plant raw material.

The wheat germs are added to an extraction medium in an amount of 15%. The extraction medium contains, as enzymes, cellulases and hemicellulases (Viscozyme from Novozymes) at a dose of 0.1% by weight.

The initial pH is 11. It stabilizes at about 7 after a few minutes.

After rapid stirring in order to disperse the mixture, the mixture is maintained with slow stirring for 2 hours at 50° C.

The mixture is then put through a centrifugal settler at 2 500 g.

The crude extract is subjected to the action of protease (Flavourzyme from Novozymes) at 0.1% by weight at a pH of about 7 for 2 hours at 50° C., with slow stirring.

Separation is then carried out by tangential filtration with a membrane having a 50 Kd cut-off of 0.2 μm.

The purified extract is immediately frozen and subsequently free-dried.

A beige powder having the following composition is obtained:

| | | |
|---|---|---|
| DNA | 0.13% | by weight |
| RNA | 0.59% | by weight |
| Carbohydrates | 60% | by weight |
| Proteins | 28% | |
| Ash (minerals) | 8.4% | |
| Lipids | <0.1% | |
| Vitamin B | | |
| (B₁, B₂, B₆, B₉) | 15 | mg/100 g |

Tests

The efficacy of the wheat germ DNA extract (dry extract at 0.13% of DNA) was tested on a monolayer culture of normal human dermal fibroblasts subjected or otherwise to UVB irradiation (250 mJ/cm²). The concentration tested is 2.26 μg/ml of DNA, a noncytotoxic concentration.

The fibroblasts are inoculated (passage P6) into 24-well plates at the rate of 70 000 cells/well and allowed to incubate in an incubator for 24 hours at 37° C., 5% CO₂ and 95% humidity. The culture medium is composed of Dulbecco's Modified Eagle Medium without phenol red containing 4.5 g/l of glucose, 10% of neonate calf serum, 100 U/ml of penicillin, 100 μg/ml of streptomycin, 2 mM of glutamine and 1% of a solution of nonessential amino acids.

Test 1

Determination of the effect of the wheat germ DNA extract on protection of normal human dermal fibroblasts subjected to UVB radiation

The cells are irradiated and treated for 24 hours after inoculation. The cells are irradiated at 250 mJ/cm² of UVB and the extract to be tested is brought into contact with the cells during irradiation at the concentration of 2.26 μg/ml of DNA. In the same manner, pure DNA is tested at the same concentration. The extract to be tested and the pure DNA are diluted in HBSS phosphate buffer.

In parallel, a positive control (nonirradiated cells) and a negative control (irradiated but nontreated cells) are prepared.

After irradiation, the cells are rinsed twice with HBSS and then incubated for 24 hours in the culture medium defined above.

Protection of the fibroblasts is evaluated by quantifying the metabolic activity of the mitochondrial dehydrogenases of the cells by measuring hydrolysis of MTT (3-{4,5-dimethylthiazol-2-yl}-2,5-diphenyltetra-zolium bromide).

The measurements are carried -out 24 hours after irradiation. The MTT solution is incubated for 3 hours at 37° C. The MTT is then removed and dimethyl sulfoxide is deposited in each well. After stirring for 5 minutes, the reading is carried out at 570 nm.

The results relating to the effect of the wheat germ DNA extract on cell protection are:

| Test | % cell viability |
|---|---|
| Nonirradiated control | 100% |
| Irradiated control | 51% |
| Irradiated with pure DNA | 78% |
| Irradiated with extract | 105% |

The wheat germ DNA extract, at the concentration of 2.26 μg/ml, causes a very significant protection of the fibroblasts against UVB radiation. Indeed, a cellular protection of 47% is observed compared with the irradiated but nontreated cells.

Test 2

Determination of the effect of the wheat germ DNA extract on the regeneration of normal human dermal fibroblasts subjected to UVB radiation

The cells are irradiated and treated 24 hours after inoculation. The cells are incubated in HBSS phosphate buffer and irradiated at 250 mJ/cm² of UVB. After irradiation, the cells are rinsed twice with HBSS and then incubated for 24 hours with the extract to be tested at the concentration of 2.26 μg/ml in the culture medium defined above. In the same manner, pure DNA is tested at the same concentration.

In parallel, a positive control (nonirradiated cells) and a negative control (irradiated but nontreated cells) are prepared.

The regeneration of the fibroblasts is evaluated -by quantifying the metabolic activity of the mitochondrial dehydrogenases of the cells by measuring hydrolysis of MTT.

The measurements are carried out 24 hours after the irradiation. The MTT solution is incubated for 3 hours at 37° C. The MTT is then removed and dimethyl sulfoxide is deposited in each well. After stirring for 5 minutes, the reading is carried out at 570 nm.

The results relating to the effect of the wheat germ DNA extract on cell regeneration are:

| Test | % cell viability |
|---|---|
| Nonirradiated control | 100% |
| Irradiated control | 65% |
| Irradiated with pure DNA | 65% |
| Irradiated with extract | 93% |

The wheat germ DNA extract, at the concentration of 2.26 μg/ml, causes a very significant regeneration of the fibroblasts after irradiation with UVB radiation. Indeed, a cell regeneration of 35% is observed compared with the irradiated but nontreated cells.

1. A method for producing an aqueous extract rich in nucleic acids from a plant material, which consists in:
extracting, in an aqueous medium, the plant material in the presence of cellulolytic enzymes at an initial pH of 9 to 13,
separating the plant material in order to recover an aqueous extract,
treating the extract with a protease,
separating the insolubles in order to recover a purified aqueous extract.

2. The method according to claim 1, wherein the plant material is a plant embryo or a seed rich in DNA or in RNA.

3. The method according to claim 1, wherein the plant material consists of wheat germs.

4. The method according to claim 1, wherein the cellulolytic enzymes are selected from pectinases and cellulases.

5. The method according to claim 1, wherein the plant material/water ratio is from 5 to 25% weight/volume.

6. The method according to claim 1, wherein the separation of the plant material from a crude aqueous extract is carried out using a centrifugal settler.

7. The method according to claim 1, wherein the separation of the insolubles is carried out using ultrafiltration or tangential microfiltration.
